(19) United States
(12) Patent Application Publication
(10) Pub. No.: US 2010/0317629 A1
(43) Pub. Date: Dec. 16, 2010

(54) DISSOLVABLE PHARMACEUTICAL IMPLANT

(75) Inventors: Genevieve L. Gallagher, Mendota Heights, MN (US); Kimberly Chaffin, Woodbury, MN (US); Zhongping Yang, Woodbury, MN (US)

Correspondence Address: Medtronic, Inc., 710 Medtronic Parkway, Minneapolis, MN, 55432, US

(73) Assignee: Medtronic, Inc., Minneapolis, MN (US)

(21) Appl. No.: 12/814,281

(22) Filed: Jun. 11, 2010

Related U.S. Application Data
(60) Provisional application No. 61/186,279, filed on Jun. 11, 2009.

(51) Int. Cl. A61K 31/65; A61P 31/00; A61P 7/04; A61P 29/00; A61P 25/04; A61N 1/00

(52) U.S. Cl. 514/154; 607/3

(57) ABSTRACT

A pharmaceutical implant may include a pharmaceutical and at least one excipient, and may be configured to be implanted in a body of a patient. The at least one excipient may dissolve after implantation of the pharmaceutical implant in the body of the patient and release the pharmaceutical. In some examples, the pharmaceutical implant includes at least two pharmaceuticals. The at least one excipient may be selected to provide a desired release profile of the pharmaceutical. For example, the pharmaceutical implant may be configured to dissolve and release the pharmaceutical over a length of time between about one day and about 30 days. In some examples, the pharmaceutical implant may be implanted in the body of the patient proximate to an implantable medical device.

This application claims the benefit of U.S. Provisional Application No. 61/186,279, entitled, “DISSOLVABLE PHARMACEUTICAL IMPLANT,” filed on Jun. 11, 2009, the entire content of which are incorporated herein by reference.

### TECHNICAL FIELD

The disclosure relates to implantable devices for pharmaceutical delivery in a body of a patient.

### BACKGROUND

Implantable medical devices (IMDs) include a variety of devices that provide therapy (such as electrical simulation or drug delivery) to a patient, monitor a physiological parameter of a patient, or both. IMDs typically include a number of functional components encased in a housing. The housing is implanted in a body of the patient. For example, the housing may be implanted in a pocket created in a torso of a patient. The housing may be constructed of a biocompatible material, such as titanium. While the housing is biocompatible, there may still be a risk of infection to the patient as a result of the implantation procedure.

### SUMMARY

In general, the disclosure is directed to a pharmaceutical implant that includes at least one pharmaceutical disposed in a dissolvable carrier. The dissolvable carrier may include at least one excipient, and may be selected to dissolve and release the pharmaceutical over a predetermined length of time. The predetermined release period may be, for example, between about 24 hours and about 30 days. In some examples, the predetermined release period may be less than about 24 hours or greater than about 30 days. The pharmaceutical implant may be implanted in a body of a patient proximate to a site at which the pharmaceutical is to be delivered, such as an infection site or a pain site.

In some examples, the pharmaceutical implant is implanted in a body of a patient substantially simultaneously with an implantable medical device (IMD). In some implementations, the pharmaceutical implant may be configured to be attached to or implanted adjacent to the IMD to, for example, reduce or substantially eliminate risk of post-implant infection at the location in the patient in which the IMD is implanted, reduce pain experienced by the patient, deliver a biological molecule such as a protein to the patient, or deliver a hemostatic agent to the patient. In other examples, the pharmaceutical implant is implanted in a body of a patient without an accompanying IMD (e.g., at the site of a wound or infection). In other words, the pharmaceutical implant may be the only device implanted at the implant location. In some examples, the pharmaceutical implant may be implanted in the body of the patient transcutaneously, e.g., via an incision or via a medical instrument such as a syringe, cannula, or the like.

In some implementations, a pharmaceutical implant that includes a dissolvable carrier may provide advantages compared to other implantable pharmaceutical delivery systems. For example, the dissolvable carrier may facilitate control of the release profile or release rate of the pharmaceutical. In systems that include a pharmaceutical mixed in a non-biodegradable polymer matrix, the release rate of the pharmaceutical may be limited by the diffusion rate of the pharmaceutical through the polymer, and can only be controlled within a relatively limited range. This may result in pharmaceutical release that is too slow for some applications. In addition, the polymer matrix may make it difficult to achieve an initial burst release of the pharmaceutical within a few hours of implant. Low levels of pharmaceutical also may remain in the polymer matrix for a significant time after implant, which may increase the risk of bacteria in the patient developing a resistance if the pharmaceutical is an antimicrobial. Further, the non-biodegradable polymer matrix remains implanted in the patient indefinitely (e.g., until it is explanted), which may be undesirable in some cases.

Similarly, in systems including a pharmaceutical disposed in a biodegradable polymer, the release rate of the pharmaceutical may be limited by the degradation rate of the biodegradable polymer. This too may result in pharmaceutical release that is too slow for some applications, and may lead to difficulty in achieving an initial burst release of the pharmaceutical within a few hours of implant.

In either of these types of systems, the release rate of the pharmaceutical may also depend on chemical interactions between the pharmaceutical and the polymer matrix. Thus, in systems including two or more pharmaceuticals, the relative release rates of the two pharmaceuticals may not be similar due to different interactions (e.g., solubilities) with the polymer matrix and/or the surrounding environment (e.g., a hydrophobic pharmaceutical that is highly soluble in a hydrophobic polymer may partition into the polymer and may never completely be released into the body). This may reduce the efficacy of the pharmaceuticals.

In other pharmaceutical delivery systems, a device may include the pharmaceutical on a coating on the surface of a substrate. Such a coating may provide a shorter release profile, but must be formulated to withstand the process used to sterilize the device. This may not be practicable for some pharmaceutical substances, which may be unstable or degrade when exposed to sterilization processes used on implantable medical devices. In addition, the coating may also poorly adhere to the substrate, which may lead to delamination of the coating during the handling of the delivery system or during the release of the pharmaceutical.

In contrast, use of a dissolvable carrier may allow greater control of the release profile (e.g., the release rate and/or the release duration). In some examples, the release profile of a pharmaceutical disposed in a dissolvable carrier may depend primarily on the dissolution rate of the dissolvable carrier, and not diffusion of the pharmaceutical through the carrier or degradation of the carrier. This may facilitate faster release of the pharmaceutical, and may allow greater latitude in selecting the release profile of the pharmaceutical from the pharmaceutical implant. For example, as described above, the pharmaceutical may be released from the implant over a period of time as little as about one day, as long as about 30 days or any length of time between about one day and about 30 days. In some embodiments, 100% of the pharmaceutical is released from the implant between 1 and 30 days, e.g., between 2 and 20 days, or between 1 and 14 days, or between 3 and 10 days, or between 3 and 7 days. In another embodiment 100% of the implant is dissolved or absorbed between 1 and 30 days, e.g., between 2 and 20 days, or between 1 and 14 days, or between 3 and 10 days or between 3 and 7 days. For example, when the pharmaceutical implant includes polyacrylic acid, hydroxypropyl cellulose, minocycline HCl, and rifampin, the 100% of the minocycline HCl and rifampin may be released or 100% of the implant may dissolve or be absorbed between 1 and 14 days, and more specifically, between 3 and 7 days.

In addition, in examples in which the pharmaceutical implant includes at least two pharmaceuticals, the release rates of the at least two pharmaceuticals may be substantially similar. The release rate of the pharmaceuticals may depend on the dissolution rate of the dissolvable carrier. In other words, chemical interactions between the pharmaceuticals and the dissolvable carrier may not play as significant a role in the release rate of the pharmaceuticals.

A pharmaceutical implant that includes a dissolvable carrier and at least one pharmaceutical also may substantially eliminate retention of any pharmaceutical in or on the pharmaceutical implant. In examples in which the pharmaceutical comprises an antimicrobial, this may mitigate or substantially eliminate a possibility of bacteria in the patient developing an antimicrobial resistance due to prolonged exposure to low levels of antimicrobial.

In one aspect, the disclosure is directed to a system that includes an implantable medical device implanted in a body of a patient and a pharmaceutical implant implanted in the body of the patient proximate to the implantable medical device. According to this aspect of the disclosure, the pharmaceutical may include at least one excipient and a pharmaceutical. In some examples, the pharmaceutical implant is configured to substantially fully dissolve within about 30 days after implantation of the pharmaceutical implant in the body of the patient.

In another aspect, the disclosure is directed to a kit that includes a pharmaceutical implant including at least one excipient and a pharmaceutical, and an implantable medical device. According to this aspect of the disclosure, the pharmaceutical implant is configured to be attached to the implantable medical device, and the implantable medical device is configured to have the pharmaceutical implant attached thereto.

In a further aspect, the disclosure is directed to a pharmaceutical implant that includes at least one excipient, minocycline, and rifampin. According to this aspect of the disclosure, the pharmaceutical implant is configured to be implanted in a body of a patient and substantially fully dissolve within about 30 days of implantation.

In an additional aspect, the disclosure is directed to a method that includes implanting a pharmaceutical implant in a body of a patient. According to this aspect of the disclosure, the pharmaceutical implant is configured to substantially fully dissolve within about 30 days after implantation of the pharmaceutical implant in the body of the patient.

The details of one or more examples of the disclosure are set forth in the accompanying drawings and the description below. Other features, objects, and advantages of the disclosure will be apparent from the description and drawings, and from the claims.

### BRIEF DESCRIPTION OF DRAWINGS

FIG. 1 is a conceptual diagram that illustrates an example of a therapy system that may be used to provide cardiac stimulation therapy to a patient, and which includes a pharmaceutical implant attached to an implantable cardiac device.

FIG. 2 is a conceptual diagram that illustrates an example of a therapy system that may be used to provide cardiac stimulation therapy to a patient, and which includes a pharmaceutical implant attached to a cardiac lead.

FIG. 3 is a conceptual diagram that illustrates an example of a pharmaceutical implant implanted in an abdomen of a patient.

FIG. 4 is a cross-sectional diagram that illustrates an exemplary pharmaceutical implant adhered to a housing of an implantable cardiac device.

FIG. 5 is a cross-sectional diagram of an exemplary system that includes an antimicrobial accessory disposed in a depression formed in an exterior of an implantable cardiac device.

FIG. 6 is a flow diagram that illustrates an example technique for forming a pharmaceutical implant.

### DETAILED DESCRIPTION

In general, the disclosure is directed to a pharmaceutical implant that includes at least one pharmaceutical disposed in a dissolvable carrier. The dissolvable carrier may include at least one excipient, and may be selected to dissolve and release the pharmaceutical over a predetermined length of time. The predetermined release period may be, for example, between about 24 hours (one day) and about 30 days. The predetermined release period also may be less than about 24 hours or greater than about 30 days. In some examples, 100% of the pharmaceutical is released from the implant between 1 and 30 days, e.g., between 2 and 20 days, or between 1 and 14 days, or between 3 and 10 days, or between 3 and 7 days. In other examples, 100% of the implant is dissolved or absorbed between 1 and 30 days, e.g., between 2 and 20 days, or between 1 and 14 days, or between 3 and 10 days and or between 3 and 7 days. For example, when the pharmaceutical implant includes polyacrylic acid, hydroxypropyl cellulose, minocycline HCl, and rifampin, the 100% of the minocycline HCl and rifampin may be released or 100% of the implant may dissolve or be absorbed between 1 and 14 days, and more specifically, between 3 and 7 days.

In some examples, the pharmaceutical implant is implanted in a body of a patient proximate to an infection site or a site at which infection is predicted or likely to occur. For example, the pharmaceutical implant may be implanted in a body of a patient substantially simultaneously with an implantable medical device (IMD), or may be implanted in a body of a patient without an accompanying IMD. In other examples, the pharmaceutical implant may be implanted in a body of a patient without an accompanying IMD (e.g., at the site of a wound or infection). In other words, the pharmaceutical implant may be the only device implanted at the implant location of the pharmaceutical implant. In some examples, the pharmaceutical implant may be implanted in the body of the patient transcutaneously, e.g., via an incision or via a medical instrument such as a syringe, cannula, or the like.

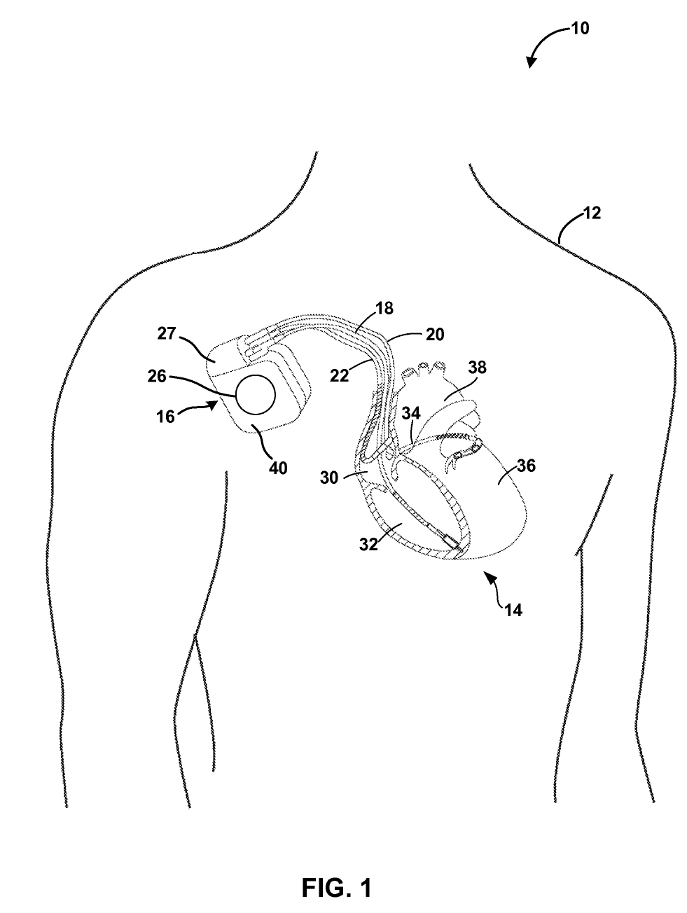

FIG. 1 is a conceptual diagram illustrating an example of a therapy system 10 that may be used to provide therapy to a patient 12. Patient 12 ordinarily, but not necessarily, will be a human. Therapy system 10 includes an implantable cardiac device (ICD) 16 connected (or “coupled”) to leads 18, 20, and 22. ICD 16 may be, for example, a device that provides cardiac rhythm management therapy to heart 14, and may include, for example, an implantable pacemaker, cardioverter, and/or defibrillator that provides therapy to heart 14 of patient 12 via electrodes coupled to one or more of leads 18, 20, and 22. In the example illustrated in FIG. 1, ICD 16 has a pharmaceutical implant 26 attached to a surface of a housing 40 of ICD 16.

Leads 18, 20, 22 that are coupled to ICD 16 extend into the heart 14 of patient 12 to sense electrical activity of heart 14 and/or deliver electrical stimulation to heart 14. In the example shown in FIG. 1, right ventricular (RV) lead 18 extends through one or more veins (not shown), the superior vena cava (not shown), and right atrium 30, and into right ventricle 32. Left ventricular (LV) coronary sinus lead 20 extends through one or more veins, the vena cava, right atrium 30, and into the coronary sinus 34 to a region adjacent to the free wall of left ventricle 36 of heart 14. Right atrial (RA) lead 22 extends through one or more veins and the vena cava, and into the right atrium 30 of heart 14. In other examples, ICD 16 may deliver stimulation therapy to heart 14 by delivering stimulation to an extravascular tissue site in addition to or instead of delivering stimulation via electrodes of intravascular leads 18, 20, 22.

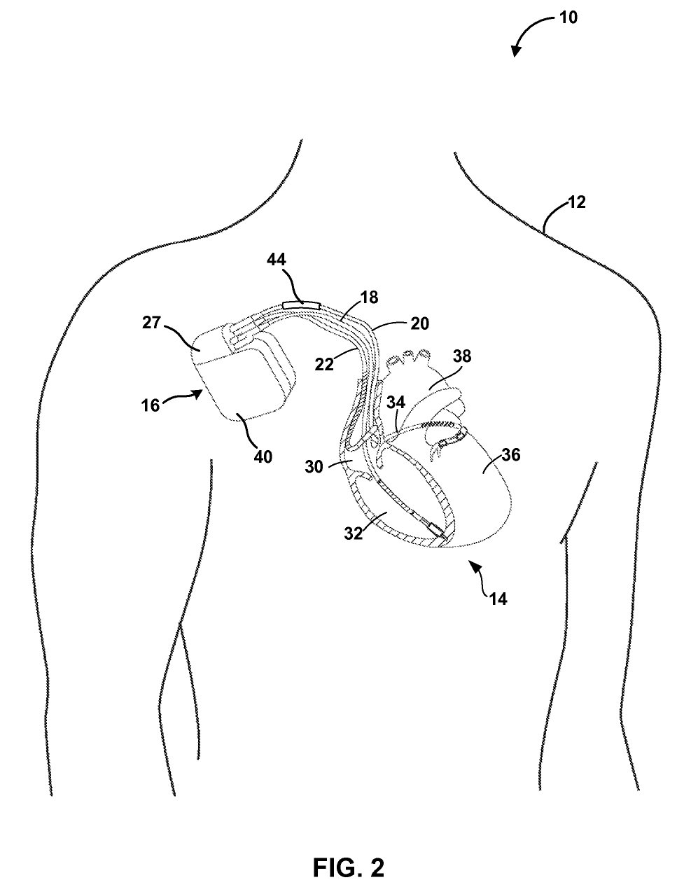

While the examples in this disclosure are primarily directed to a pharmaceutical implant 26 attached to an ICD 16, in other examples, pharmaceutical implant 26 may be attached to or implanted proximate to another IMD. For example, pharmaceutical implant 26 may be attached to or implanted proximate to an implantable drug delivery device, an implantable monitoring device that monitors one or more physiological parameter of patient 12, an implantable neurostimulator (e.g., a spinal cord stimulator, a deep brain stimulator, a pelvic floor stimulator, a peripheral nerve stimulator, or the like), a gastric stimulator, a stimulator for control or management of urinary or fecal incontinence, a cardiac or neurological lead, a catheter, an orthopedic device such as a spinal device or bone plate, a stent, a vascular graft, a hydrocephalus shunt, an ear implant, a nose implant, a throat implant, or the like. For example, as FIG. 2 illustrates, a pharmaceutical implant 44 may be configured as a tube or hollow cylinder that is disposed around a cardiac lead 20. In general, pharmaceutical implant 26 may be attached to or implanted proximate to any medical device configured to be implanted in a body of a patient 12.

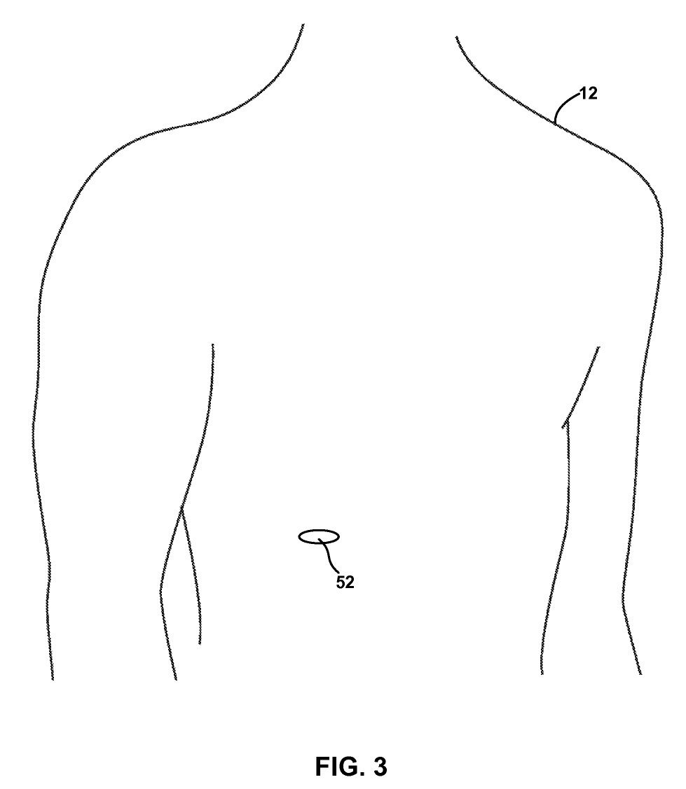

In other examples, as described below with reference to FIG. 3, a pharmaceutical implant 52 may not be used with an implantable medical device, such as ICD 16, and may instead be implanted in patient 12 without an accompanying implantable medical device. Pharmaceutical implant 52 may be implanted anywhere within the body of patient 12. For example, pharmaceutical implant 52 may be implanted proximate to a site of an infection in the body of patient 12 or a site at which pain medication is to be delivered. In the example illustrated in FIG. 3, pharmaceutical implant 52 is implanted in an abdomen of patient 12. In some examples, pharmaceutical implant 26, 44, 52 may be implanted in the body of the patient transcutaneously, e.g., via an incision or via a medical instrument such as a syringe, cannula, or the like.

Returning now to FIG. 1, pharmaceutical implant 26 may be attached to a surface of housing 40 and/or connector block 27 of ICD 16 and includes a dissolvable carrier and at least one pharmaceutical. As described above, in some examples, pharmaceutical implant 26 may reduce or substantially eliminate the risk of post-implant infection proximate to the implant location of ICD 16 by releasing an antimicrobial subsequent to implantation of ICD 16 and pharmaceutical implant 26 in the body of patient 12. In other examples, pharmaceutical implant 26 may reduce pain experienced by patient 12 due the implantation procedure by releasing an analgesic, such as a pain medication or anti-inflammatory agent, at the implant site, or may deliver hemostatic agents to reduce internal bleeding after implantation of ICD 16. Pharmaceutical implant 26 also may release a protein such as insulin, siRNA to treat genetic conditions, a chemotherapy drug, or another genetic modifier.

The at least one pharmaceutical in antimicrobial implant 26 may include, for example, an analgesic, such as a pain medication or anti-inflammatory agent, a hemostatic agent, an antimicrobial such as an antibiotic, an antiseptic, an antimicrobial peptide, a quaternary ammonium, a heavy metal or heavy metal salt, or the like. Exemplary hemostatic agents include, but are not limited to, styptics, antifibrinolytics, vitamin K, blood coagulation factors, fibrinogen, thrombin, collagen, polysaccharides, chitosan, or the like. Exemplary analgesics include, but are not limited to, pain relievers, opioids, narcotics, morphine, tramadol, acetaminophen, anti-inflammatory agents, COX-1-inhibitors, COX-2-inhibitors, aspirin, ibuprofen, naproxen, natural herbal compounds, steroids, or the like. Examples of antibiotic classes include fluoroquinolones, aminoglycosides, lincosamides, macrolides, tetracyclines, florochinolones, glycopeptides, and penicillins. Exemplary antibiotics include minocycline, clindamycin, rifampin, tigecycline, daptomycin, gentamicin, netilmicin, neomycin, amikacin, kanamycin, erythromycin, tetracycline, ciprofloxacin, and teicoplanin. In some examples, the antimicrobial may be provided in a salt form, e.g., minocycline HCl, may be lyophilized, or may be converted into a fatty-acid salt. For example, gentamicin may be reacted with a sodium dodecyl sulfate, sodium palmitate, and sodium myristate to form gentamicin pentakis(dodecylsulfate), gentamicin petakis(malmitate), and gentamicin pentakis(myristate), respectively. Other antibiotics may also be reacted with fatty acids such as sodium dodecyl sulfate, sodium palmitate, or sodium myristate to form antibiotic fatty-acid salts. In addition, other fatty acids may be used. As will be understood by one of ordinary skill in the art, these lists of exemplary antimicrobials, antibiotic classes and antibiotics are not exhaustive, and the techniques described in the present disclosure may be adapted to other antimicrobials, antibiotic classes, antibiotics, or other pharmaceutical classes without departing from the spirit or scope of the disclosure.

The at least one pharmaceutical may be selected to provide efficacious therapy (e.g., pain relief, infection prevention, hemostatis) proximate to the implant site at which pharmaceutical implant 26 is implanted, e.g., the pocket in which ICD 16 and pharmaceutical implant 26 are implanted. In some examples, pharmaceutical implant 26 may include at least two pharmaceuticals, e.g., a first pharmaceutical and a second pharmaceutical different than the first pharmaceutical. For example, the first pharmaceutical may include a pain medication and the second pharmaceutical may include a hemostatic agent. Other combinations of first and second pharmaceuticals will be apparent to one of ordinary skill in the art and are within the scope of this disclosure.

In other examples, pharmaceutical implant 26 may include at least two antimicrobials that are selected to provide efficacious prevention or treatment of any infection that may be present proximate to the implant site at which pharmaceutical implant 26 is implanted, e.g., an infection in the pocket in which ICD 16 and pharmaceutical implant 26 are implanted. In some examples, pharmaceutical implant 26 may include at least two antimicrobials, e.g., a first antimicrobial and a second antimicrobial different than the first antimicrobial, and the combination of the at least two antimicrobials may be selected to efficaciously treat or prevent any infection present proximate to the implant site of the ICD 16. In some examples, the at least two antimicrobials include minocycline and rifampin.

Pharmaceutical implant 26 may further include a dissolvable carrier, which includes at least one excipient. The at least one excipient may be selected to provide desired properties to pharmaceutical implant 26. For example, the at least one excipient may be selected to provide the desired release profile of the pharmaceutical (e.g., both the release rate and the release duration). The at least one excipient also may be selected to provide other properties, such as adhesiveness, shelf life, pharmaceutical stability, or the like.

The at least one excipient may include a binder. A binder holds the ingredients in the pharmaceutical implant 26 together during storage and implantation of the implant 26. Exemplary binders include, but are not limited to, a starch, such as a pregelatinized starch; a sugar; cellulose; a modified cellulose, such as microcrystalline cellulose, hydroxypropyl cellulose, hydroxypropyl methyl cellulose, lactose, or lactose monohydrate; a sugar alcohol, such as xylitol, sorbitol, or maltitol; dibasic calcium phosphate, or the like.

In some examples, the at least one excipient also includes a disintegrant. A disintegrant expands and dissolves when exposed to water, which may cause pharmaceutical implant 26 to break apart and release the pharmaceutical. The disintegrant may include, for example, a starch, cellulose, cross-linked polyvinyl pyrrolidone, sodium starch glycolate, cross-linked sodium carboxymethyl cellulose, or the like. When pharmaceutical implant 26 includes a disintegrant, the amount and type of disintegrant may be selected to provide the desired disintegration rate, which may influence or determine the rate at which pharmaceutical implant 26 dissolves and, ultimately, the release profile of the pharmaceutical.

The at least one excipient also may include a filler or diluent. A filler or diluent may be added to pharmaceutical implant 26 to increase the volume of pharmaceutical implant 26 to a desired amount. For example, an increase in volume may facilitate production of pharmaceutical implant 26 or handling of pharmaceutical implant 26 by a user, such as a clinician or patient. The filler or diluent may include plant cellulose, dibasic calcium phosphate, lactose, sucrose, glucose, mannitol, sorbitol, calcium carbonate, magnesium stearate, or the like.

Pharmaceutical implant 26 also may include a glidant, which promotes powder flow during manufacture of pharmaceutical implant 26. In some examples, pharmaceutical implant 26 alternatively or additionally may include a lubricant or antiadherent. Either separately or in combination, the glidant, lubricant, or antiadherent reduce interparticle friction and adhesion, and reduce adhesion of the implant 26 mixture to tablet punches or dies. Exemplary glidants include, for example, colloidal silicon dioxide, talc, or the like. Exemplary lubricants and antiadherents include, for example, polyethylene glycol, talc, silicon dioxide, fats such as vegetable stearin, magnesium stearate, stearic acid, sodium stearyl fumarate, or the like.

The at least one excipient may further include a preservative, which may prevent or slow degradation of the pharmaceutical. Exemplary preservatives include, for example, antioxidants such as vitamin A, vitamin C, vitamin E, retinyl palmitate, or selenium, amino acids such as cysteine and methionine, citric acid, sodium citrate, synthetic preservatives such as methyl paraben and propyl paraben, or the like.

In some examples, the pharmaceutical implant 26 may be coated with a coating. The coating may serve to mitigate or substantially prevent components of pharmaceutical implant 26 from deteriorating. For example, certain components of pharmaceutical implant 26 may degrade in the presence of water or light. The coating may reduce or substantially prevent water vapor or light from affecting the components, and thus improve the stability or shelf life of pharmaceutical implant 26. Coatings may include, for example, cellulose, synthetic polymers, shellac, corn protein zein, other polysaccharides, or the like.

Relative amounts of the various components of pharmaceutical implant 26 may be selected based on a number of considerations. For example, one may consider the desired form factor and size of pharmaceutical implant 26, the desired shelf life, the desired release profile of the pharmaceutical, the method of implantation (e.g., injection or incision), or the like. In some examples, pharmaceutical implant 26 may include between about 5 wt. % and about 10 wt % pharmaceutical and between about 90 wt. % and about 95 wt. % excipient.

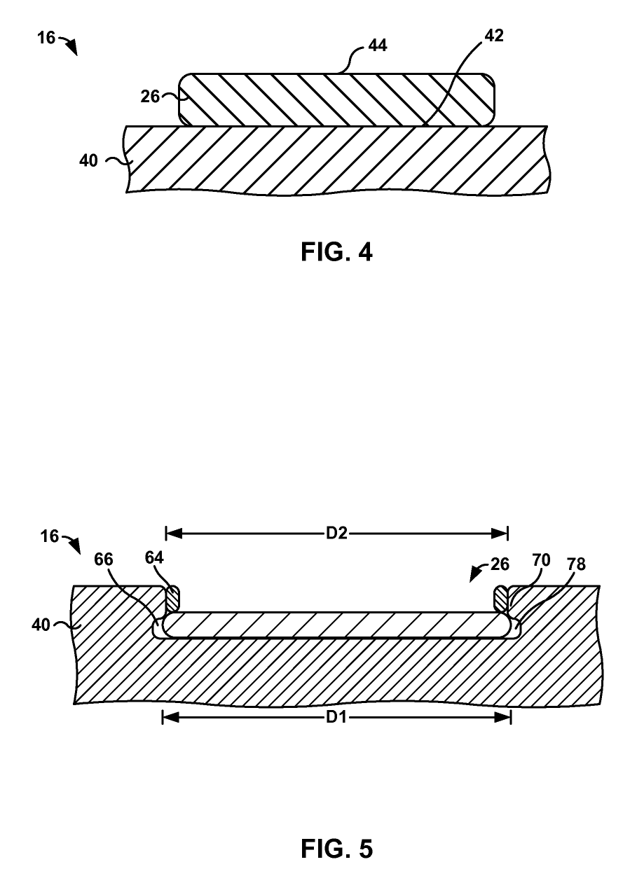

Pharmaceutical implant 26 may be formed in some examples to substantially conform to the curvature (or lack of curvature) of the housing 40 of ICD 16 or connector block 27 of IMD 16. For example, a first surface 42 of pharmaceutical implant 26, which faces housing 40, may be substantially planar, may be convex, may be concave, or may include a more complex curvature. As illustrated in FIG. 4, in some examples, a surface of housing 40 to which pharmaceutical implant 26 is to be attached may be substantially planar, and first surface 42 of pharmaceutical implant 26 may be substantially planar. Second surface 44 may be substantially planar, or may comprise some curvature. In other words, second surface 44 may substantially parallel first surface 42, or may not substantially parallel first surface 42. In other examples, a surface of housing 40 may be substantially convex, and first surface 42 may be substantially concave with a similar or substantially identical radius of curvature to the surface of housing 40. Again, second surface 44 may or may not be substantially parallel first surface 42. In other examples, pharmaceutical implant 26 may not be formed to substantially conform to the curvature or lack or curvature of the housing 40 or connector block 27.

Pharmaceutical implant 26 may also include rounded edges, as shown in FIG. 4. In some examples, rounded edges may to reduce tissue irritation or erosion, or may reduce migration of pharmaceutical implant 26.

In addition, in some examples, the at least one excipient may provide adhesiveness when wet to tissue or ICD 16. For example, a pharmaceutical implant 26 including polyacrylic acid, chitosan, poly(ethylene oxide), a methylvinylether/maleic acid copolymer, a vinylpyrrolidone/vinyl acetate copolymer, polyvinylpyrrolidone, or the like may be bioadhesive when wet, and may adhere to tissue and medical devices.

Although pharmaceutical implant 26 may be self-adhesively attached to housing 40 of ICD 16 or bodily tissue, in other examples, pharmaceutical implant 26 may be attached to ICD 16 or tissue by other means. For example, pharmaceutical implant 26 may be attached to ICD 16 by a suture to connector block 27 or an aperture defined in connector block 27. In other examples, pharmaceutical implant 26 may not be attached to housing 40 in any manner, and may be implanted in patient 12 proximate to ICD 16 or may be implanted without an accompanying ICD 16.

In some examples, the relative shapes of the IMD and pharmaceutical implant 26 may be configured to result in a friction fit or other type of physical coupling between the IMD and pharmaceutical implant 26. For example, FIG. 2 illustrates a pharmaceutical implant 44 comprising a hollow cylinder disposed about a lead 20. The cylindrical shape of pharmaceutical implant 44 may prevent the implant 44 from disengaging from lead 20. In some examples, at least a portion of an inner surface of pharmaceutical implant 44 may contact lead 20 and form a friction fit with lead 20 or adhere to lead 20.

As another example, as shown in FIG. 5, a pharmaceutical implant 26 also may be disposed in a depression 66 formed in housing 40 (or connector block 27). Depression 66 may be sized to fit at least a portion of pharmaceutical implant 26. Disposed on the wall 70 of depression 66 is a grommet 74, which may be formed of, for example, a biocompatible polymer such as silicone rubber. Grommet 74 may assist in mechanically securing pharmaceutical implant 26 in depression 66. In some embodiments, wall 70 may not have a grommet 64 attached thereto, and pharmaceutical implant 26 may be mechanically secured within depression 66 by a friction fit with wall 70 of depression 66, or may fit within depression 66 without contacting the wall 70 of depression 66. In any of these examples, pharmaceutical implant 26 may adhere to housing 40 (e.g., implant 26 may be self-adhering when wet).

Wall 70 may include an undercut 68, which may facilitate retention of pharmaceutical implant 26 in depression 66. For example, pharmaceutical implant 26 may have a diameter D1 substantially equal to or greater than the diameter D2 of depression 66. Pharmaceutical implant 26 may then be pressed or snapped into depression 66 such that a portion of pharmaceutical implant 26 is disposed in the recession formed by undercut 68. In this way, undercut 68 may substantially restrain pharmaceutical implant 26 in depression 66. In some examples, pharmaceutical implant 26 simply may be pressed within a concave feature formed in a substrate (e.g., a housing of an IMD). For example, a tablet-pressing (or cold-pressing) process can be used to couple pharmaceutical implant 26 to a device or depression 66. An exemplary tablet-pressing operation can include forming a tablet or other form factor from the mixed components of the pharmaceutical implant by application of mechanical pressure without application of heat or additional solvent processing. The amount of time, pressure, and other parameters used will vary by the form factor and/or composition of the pharmaceutical implant.

Pharmaceutical implant 26 may be configured in a variety of form factors, including, for example, a tablet, a capsule, a caplet, a sphere, a rod, a film, a coating, a disk, a sheet, a hollow cylinder, or the like. In some examples, a coating of the at least one excipient and the pharmaceutical may be deposited by a powder deposition process on a substrate, such as, for example, a housing of an IMD. The coating may then dissolve after implantation of the IMD to release the pharmaceutical. The form factor may be selected based on, for example, ease of manufacture or handling after manufacture, the shape of the IMD to which pharmaceutical implant 26 is to be attached, or the like.

Pharmaceutical implant 26 may include a range of thicknesses, such as, for example, between about 0.001 inch and about 0.05 inch. In addition, pharmaceutical implant 26 may have a width between about 0.25 inches and about 1.0 inches. In some examples, pharmaceutical implant 26 may include dimensions outside of those described herein. The dimensions of pharmaceutical implant may be selected to control the surface area to volume ratio of the implant 26, which may affect the rate of dissolution of implant 26.

In any of the examples described above, the amount of pharmaceutical may vary widely. In any case, the amount of pharmaceutical may be selected to provide a therapeutically efficacious concentration of pharmaceutical at or proximate to the implant site of pharmaceutical implant 26 in the body of patient 12 shortly after implantation of pharmaceutical implant 26 in patient 12 (e.g., within 4 hours). Other considerations influencing the amount of pharmaceutical in pharmaceutical implant 26 include, for example, a time over which elution may continue or a minimum amount of pharmaceutical to be eluted within a certain time after implant. For example, elution of pharmaceutical from pharmaceutical implant 26 may be desired to continue for between about one day and about 30 days after implant of pharmaceutical implant 26 in the body of patient 12, and the amount of pharmaceutical in pharmaceutical implant 26 may be selected to provide the desired elution profile.

In some examples, pharmaceutical implant 26 may be packaged in a foil package or other substantially air and water impermeable package that is vacuum sealed or backfilled with an inert gas. Pharmaceutical implant 26 may then be sterilized by, for example, electron beam sterilization, gamma beam sterilization, ethylene oxide sterilization, autoclaving, or the like.

In some examples, pharmaceutical implant 26 may be packaged together with an ICD 16 at the time of manufacture, e.g., in a shipping box in which ICD 16 and pharmaceutical implant 26 are shipped to a distributor. In other examples, pharmaceutical implant 26 may be packaged with an ICD 16 at the distributor prior to being shipped to a sales representative or clinician, or in the hospital or lab prior to transport to the operating room. This flexibility in packaging pharmaceutical implant 26 and ICD 16 may facilitate sale or use of pharmaceutical implant 26 only in cases where a pharmaceutical implant 26 is needed, and may allow pharmaceutical implant 26 to be an economical product for end users, e.g., clinicians and, ultimately, patients.

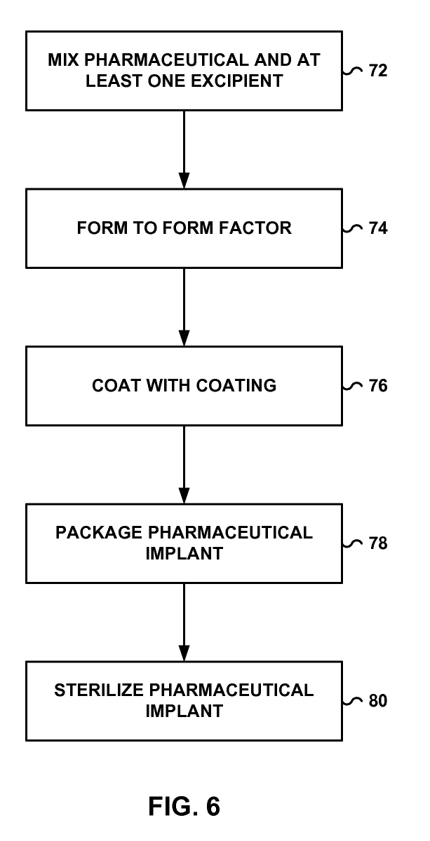

FIG. 6 is a flow diagram of an example of a method for forming a pharmaceutical implant 26. Initially, a pharmaceutical and at least one excipient are mixed (72). In some examples, the pharmaceutical and the at least one excipient may be mixed as dry powders and formed into a desired form factor (74). For example, the dry powder may be compression molded, tablet pressed, or the like. In other examples, the pharmaceutical and the at least one excipient may require granulation before being formed into the desired form factor. The granulation process may include dry granulation, wet granulation, or fluidized bed granulation. Once the components have been granulated, the grains may be formed into a desired form factor (74) by, for example, compression molding, tablet pressing, or the like.

Once the mixture is in the desired form factor, the mixture may optionally be coated (76). As described above, the coating may protect the mixture from water vapor or light, and may increase the shelf-life of the pharmaceutical implant 26. A coating may also contribute to control of the release profile of the pharmaceutical.

The antimicrobial accessory 26 then may be packaged (78). For example, pharmaceutical implant 26 may be packaged in a foil pouch or another substantially air tight container. The foil pouch may be evacuated of air by a vacuum, or may be backfilled with an inert gas. In some examples, the foil pouch may also enclose a desiccant to trap moisture present in the foil pouch.

Pharmaceutical implant 26 then may be sterilized (80). The sterilization method may be selected to provide an efficacious sterilization of pharmaceutical implant 26 while minimizing degradation of the pharmaceutical in pharmaceutical implant 26. Exemplary sterilization methods include, for example, electron beam sterilization, gamma beam sterilization, ethylene oxide sterilization, or autoclaving.

In other embodiments, the method illustrated in FIG. 6 may include additional, optional steps. For example, one or more components of pharmaceutical implant 26 may be granulated prior to forming to the desired form factor (74). As another example, the granulated component may be microencapsulated.

Conversely, the method illustrated in FIG. 6 may not include all of the illustrated steps. For example, the pharmaceutical and at least one excipient may be sterilized prior to mixing and may be aseptically processed using sterilized equipment. The method then may not include the sterilization step (80).

### Example

### Example 1

A pharmaceutical implant 26 will include about 80 mg crosslinked polyacrylic acid, about 40 mg hydroxypropyl cellulose, about 10 mg minocycline HCl, and about 10 mg rifampin. The components of the pharmaceutical implant 26 will be dry-mixed together and compression molded to form a tablet or disk. Dry mixing and compression molding do not use a solvent or elevated temperatures, and may help maintain drug purity and minimize drug degradation. The pharmaceutical implant 26 will be packaged in a vacuum-evacuated aluminum foil package and sterilized by cold electron-beam sterilizing.

Such a pharmaceutical implant 26 is expected to be adhesive when wet due to the polyacrylic acid. Pharmaceutical implant 26 is expected to adhere to tissue and metallic or polymeric substrates, such as the housing 40 or connector block 27 of ICD 16.

Pharmaceutical implant 26 is also expected to provide steady release (e.g., zero order release kinetics, or substantially constant release) of the minocycline HCl and rifampin over about 24 to about 72 hours. The pharmaceutical implant 26 is expected to substantially fully dissolve within about 7 days, releasing substantially 100% of the minocycline HCl and rifampin within this time.

In addition, the polyacrylic acid is expected to stabilize the minocycline HCl and rifampin and provide a shelf life of about 2 years at room temperature.

### Example 2

A first formulation for a pharmaceutical implant was prepared, targeting relatively quick in-vitro dissolution. The first formulation included the components shown in Table 1. Column 1 names the components, column 2 lists a manufacturer of the component, column 3 lists the function of the component within the pharmaceutical implant, column 4 lists the weight percentage of each component, and column 5 lists the absolute amount of each component in a 100 milligram tablet made from the first formulation.

TABLE 1

| | | | Weight | |
|---|---|---|---|---|
| | | | Percentage | Amount |
| Component | Manufacturer | Function | (% w/w) | (mg) |
| Rifampin | Lupin | Active | 10 | 10 |
| | Pharmaceuticals, | Ingredient | | |
| | Inc., Mumbia, India | | | |
| Minocycline | Hovione, Loures, | Active | 10 | 10 |
| | Portugal | Ingredient | | |
| PanExcea | Mallinckrodt | Filler and | 54 | 54 |
| | Baker, Inc., | Disintegrant | | |
| | Phillipsburg, NJ | | | |
| Micro- | FMC Corp., | Filler | 10 | 10 |
| crystalline | Philadelphia, PA | | | |
| Cellulose | | | | |
| Magnesium | Mallinckrodt | Lubricant | 1 | 1 |
| Stearate | Baker, Inc., | | | |
| | Phillipsburg, NJ | | | |
| Crospovidone | International | Disintegrant | 15 | 15 |
| | Specialty | | | |
| | Products, Inc., | | | |
| | Wayne, NJ | | | |
| Total | | | 100 | 100 |

A mixture of the components listed in Table 1 was prepared as follows. First, rifampin, minocycline and microcrystalline cellulose were mixed for about 5 minutes at about 30 revolutions-per-minute (rpm) in an eight (8) quart V-blender mixer, available from Keith Machinery Corp., Lindenhurst, N.Y. Crospovidone (polyvinylpolypyrrolidone) was then added to the mixture in the V-blender, and the resulting mixture was mixed for about 5 minutes at about 30 rpm. Pan Excea was added to the mixture in the V-blender, and the resulting mixture was mixed for about 5 minutes at about 30 rpm. Finally, magnesium stearate was added to the mixture in the V-blender, and the resulting mixture was mixed for about 5 minutes at about 30 rpm. The final mixture was then compressed into tablets weighing about 100 mg using a twenty (20) station rotary tablet press with B-tooling, available from Jenn Chiang Machinery Co., Ltd., Feng-Yuan, Taiwan. Tablet hardness was between about 5 kiloponds (kp) and about 7 kp. Settings on the tablet press were controlled to achieve a weight of about 100 mg and a hardness between about 5 kp and about 7 kp.

### Example 3

A second formulation for a pharmaceutical implant was prepared, targeting a relatively quick in-vitro dissolution time, although a longer dissolution time than Example 2. The second formulation included the components shown in Table 2. Column 1 names the components, column 2 lists the weight percentage of each component, and column 3 lists the absolute amount of each component in a 100 milligram tablet made from the first formulation.

TABLE 2

| | | | Weight | |
|---|---|---|---|---|
| | | | Percentage | Amount |
| Component | Manufacturer | Function | (% w/w) | (mg) |
| Rifampin | Lupin | Active | 10 | 10 |
| | Pharmaceuticals, | Ingredient | | |
| | Inc., Mumbia, India | | | |
| Minocycline | Hovione, Loures, | Active | 10 | 10 |
| | Portugal | Ingredient | | |
| Lactose | MEGGLE | Filler | 51 | 51 |
| Monohydrate | Wasserburg GmbH, | | | |
| | Wasserburg, | | | |
| | Germany | | | |
| Micro- | FMC Corp., | Filler | 20 | 20 |
| crystalline | Philadelphia, PA | | | |
| Cellulose | | | | |
| Magnesium | Mallinckrodt | Lubricant | 1 | 1 |
| Stearate | Baker, Inc., | | | |
| | Phillipsburg, NJ | | | |
| Croscarmellose | DMV Fonterra | Disintegrant | 8 | 8 |
| Sodium | Excipients, | | | |
| | Goch, Germany | | | |
| Total | | | 100 | 100 |

A mixture of the components listed in Table 2 was prepared as follows. First, rifampin, mincycline and microcrystalline cellulose were mixed for about 5 minutes at about 30 rpm in an eight (8) quart V-blender mixer, a tumble mixer. Lactose monohydrate was then added to the mixture in the V-blender, and the resulting mixture was mixed for about 5 minutes at about 30 rpm. Croscarmellose sodium was added to the mixture in the V-blender, and the resulting mixture was mixed for about 5 minutes at about 30 rpm. Finally, magnesium stearate was added to the mixture in the V-blender, and the resulting mixture was mixed for about 5 minutes at about 30 rpm. The final mixture was then compressed into tablets weighing about 100 mg using a twenty (20) station rotary tablet press with B-tooling. Tablet hardness was between about 5 kp and about 7 kp. Settings on the tablet press were controlled to achieve a weight of about 100 mg and a hardness between about 5 kp and about 7 kp.

### Example 4

A third formulation for a pharmaceutical implant was prepared, targeting a slower in-vitro dissolution time (e.g., slower than the dissolution times targeted in Examples 2 and 3). Hydroxypropyl methyl cellulose is a hydrophilic polymer that contributed to the slower in-vitro dissolution time. The third formulation included the components shown in Table 3. Column 1 names the components, column 2 lists the weight percentage of each component, and column 3 lists the absolute amount of each component in a 100 milligram tablet made from the first formulation.

TABLE 3

| | | | Weight | |
|---|---|---|---|---|
| | | | Percentage | Amount |
| Component | Manufacturer | Function | (% w/w) | (mg) |
| Rifampin | Lupin | Active | 10 | 10 |
| | Pharmaceuticals, | Ingredient | | |
| | Inc., Mumbia, India | | | |
| Minocycline | Hovione, Loures, | Active | 10 | 10 |
| | Portugal | Ingredient | | |
| Lactose | MEGGLE | Filler | 34 | 34 |
| Monohydrate | Wasserburg GmbH, | | | |
| | Wasserburg, | | | |
| | Germany | | | |
| Hydroxypropyl | Shin Etsu Chemical | Filler | 40 | 40 |
| Methyl | Co., Ltd., Tokyo, | | | |
| Cellulose | Japan | | | |
| Pregelatinized | Colorcon, Inc., | Binder | 0.5 | 0.5 |
| Starch | Harleysville, PA | | | |
| Sodium Lauryl | Sigma-Aldrich Co., St. | Lubricant | 5 | 5 |
| Sulfate | Louis, MO | | | |
| Magnesium | Mallinckrodt | Lubricant | 0.5 | 0.5 |
| Stearate | Baker, Inc., | | | |
| | Phillipsburg, NJ | | | |
| Total | | | 100 | 100 |

A mixture of the components listed in Table 3 was prepared as follows. First, rifampinand sodium lauryl sulfate were mixed for about 5 minutes at about 30 rpm in an eight (8) quart V-blender mixer, a tumble mixer. Lactose monohydrate, minocycline, and hydrosypropyl methyl cellulose was then added to the mixture in the V-blender, and the resulting mixture mixed for about 5 minutes at about 30 rpm. Pregelatinized starch was added to the mixture in the V-blender, and the resulting mixture was mixed for about 5 minutes at about 30 rpm. Finally, magnesium stearate was added to the mixture in the V-blender, and the resulting mixture was mixed for about 5 minutes at about 30 rpm. The final mixture was then compressed into tablets weighing about 100 mg using a twenty (20) station rotary tablet press with B-tooling. Tablet hardness was between about 5 kp and about 7 kp. Settings on the tablet press were controlled to achieve a weight of about 100 mg and a hardness between about 5 kp and about 7 kp.

### Example 5

Tablets with a composition as listed in Table 1 were compressed using a single punch press. Weight variation was significant. Table 4 shows content uniformity based on a quantitative assay performed using HPLC and other standard methods.

TABLE 4

| | Minocycline | Rifampin |
|---|---|---|
| | assay | assay |
| | (%) | (%) |
| Tablet 1 | 87.6 | 91.2 |
| Tablet 2 | 89.6 | 83.0 |
| Tablet 3 | 84.9 | 91.8 |
| Tablet 4 | 86.9 | 87.1 |
| Tablet 5 | 86.2 | 92.5 |
| Tablet 6 | 86.5 | 92.1 |
| Average | 87.0 | 89.6 |

Tables 5-7 illustrate results of dissolution tests of tablets that included the composition shown in Table 1 in a 10 mM sodium phosphate buffer solution with a pH of about 6.0. Table 5 shows results after about 15 minutes, Table 6 shows results after about 30 minutes, and Table 7 shows results after about 35 minutes. As Tables 5-7 show, rifampin release was generally slower than minocycline release.

TABLE 5

| | Minocycline | Rifampin |
|---|---|---|
| | release | release |
| | (%) | (%) |
| Tablet 1 | 77.1 | 59.4 |
| Tablet 2 | 82.1 | 53.9 |
| Tablet 3 | 81.8 | 57.7 |
| Tablet 4 | 70.6 | 56.0 |
| Tablet 5 | 80.1 | 55.9 |
| Tablet 6 | 79.1 | 49.6 |
| Average | 78.5 | 55.4 |

TABLE 6

| | Minocycline | Rifampin |
|---|---|---|
| | release | release |
| | (%) | (%) |
| Tablet 1 | 82.4 | 75.4 |
| Tablet 2 | 83.6 | 67.8 |
| Tablet 3 | 83.6 | 70.1 |
| Tablet 4 | 85.2 | 70.5 |
| Tablet 5 | 86.1 | 69.4 |
| Tablet 6 | 86.8 | 62.3 |
| Average | 84.6 | 69.3 |

TABLE 7

| | Minocycline | Rifampin |
|---|---|---|
| | release | release |
| | (%) | (%) |
| Tablet 1 | 80.4 | 86.8 |
| Tablet 2 | 84.2 | 78.6 |
| Tablet 3 | 88.2 | 84.4 |
| Tablet 4 | 79.1 | 82.0 |
| Tablet 5 | 84.7 | 78.8 |
| Tablet 6 | 81.5 | 74.7 |
| Average | 83.0 | 81.1 |

### Example 6

Tablets with a composition as listed in Table 1 were compressed using a rotary tablet press, as described with respect to Example 2. Table 8 shows content uniformity based on a quantitative assay performed using HPLC and other standard methods. Assay results for both minocycline and rifampin resulted in recovery above the theoretical maximum of 100%.

TABLE 8

| | Minocycline | Rifampin |
|---|---|---|
| | assay | assay |
| | (%) | (%) |
| Tablet 1 | 111.3 | 113.1 |
| Tablet 2 | 100.9 | 106.2 |
| Tablet 3 | 118.1 | 125.8 |
| Average | 110.1 | 115.0 |

Tables 9-11 illustrate results of dissolution tests of tablets that included the composition shown in Table 1 in a 10 mM sodium phosphate buffer solution with a pH of about 6.0. Table 9 shows results after about 15 minutes, Table 10 shows results after about 30 minutes, and Table 11 shows results after about 35 minutes. As Tables 8-11 show, rifampin release was generally slower than minocycline release.

TABLE 9

| | Minocycline | Rifampin |
|---|---|---|
| | release | release |
| | (%) | (%) |
| Tablet 1 | 94.7 | 75.8 |
| Tablet 2 | 103.4 | 66.4 |
| Tablet 3 | 93.5 | 76.8 |
| Average | 97.2 | 73.0 |

TABLE 10

| | Minocycline | Rifampin |
|---|---|---|
| | release | release |
| | (%) | (%) |
| Tablet 1 | 99.1 | 94.1 |
| Tablet 2 | 107.4 | 93.0 |
| Tablet 3 | 95.6 | 94.4 |
| Average | 100.7 | 93.8 |

TABLE 11

| | Minocycline | Rifampin |
|---|---|---|
| | release | release |
| | (%) | (%) |
| Tablet 1 | 99.1 | 101.2 |
| Tablet 2 | 109.6 | 97.8 |
| Tablet 3 | 97.6 | 100.0 |
| Average | 102.1 | 99.7 |

Although the percentage released for each of minocycline and rifampin in the dissolution tests was close to 100%, the percentage released was still about 10% to about 15% lower compared to assay results. Rifampin again shows slower release than minocycline.

### Example 7

Tablets with a composition as listed in Table 2 were compressed using a single punch press. Weight variation was significant. Table 12 shows content uniformity based on a quantitative assay performed using HPLC and other standard methods.

TABLE 12

| | Minocycline | Rifampin |
|---|---|---|
| | assay | assay |
| | (%) | (%) |
| Tablet 1 | 93.1 | 97.4 |
| Tablet 2 | 88.7 | 102.5 |
| Tablet 3 | 90.6 | 97.3 |
| Tablet 4 | 97.2 | 99.0 |
| Tablet 5 | 93.6 | 96.5 |
| Tablet 6 | 88.1 | 91.9 |
| Average | 91.9 | 97.4 |

Tables 13-15 illustrate results of dissolution tests of tablets that included the composition shown in Table 2 in a 10 mM sodium phosphate buffer solution with a pH of about 6.0. Table 13 shows results after about 15 minutes, Table 14 shows results after about 30 minutes, and Table 15 shows results after about 35 minutes. As Tables 13-15 show, rifampin release was generally slower than minocycline release.

TABLE 13

| | Minocycline | Rifampin |
|---|---|---|
| | release | release |
| | (%) | (%) |
| Tablet 1 | 109.9 | 53.9 |
| Tablet 2 | 110.4 | 52.4 |
| Tablet 3 | 91.1 | 41.9 |
| Tablet 4 | 97.4 | 44.9 |
| Tablet 5 | 102.3 | 45.5 |
| Tablet 6 | 108.9 | 51.3 |
| Average | 103.3 | 48.3 |

TABLE 14

| | Minocycline | Rifampin |
|---|---|---|
| | release | release |
| | (%) | (%) |
| Tablet 1 | 105.8 | 72.7 |
| Tablet 2 | 106.0 | 67.6 |
| Tablet 3 | 96.0 | 49.4 |
| Tablet 4 | 101.3 | 69.9 |
| Tablet 5 | 102.1 | 58.0 |
| Tablet 6 | 106.5 | 67.6 |
| Average | 103.0 | 64.2 |

TABLE 15

| | Minocycline | Rifampin |
|---|---|---|
| | release | release |
| | (%) | (%) |
| Tablet 1 | 106.8 | 79.2 |
| Tablet 2 | 108.8 | 78.2 |
| Tablet 3 | 101.7 | 72.9 |
| Tablet 4 | 103.7 | 75.4 |
| Tablet 5 | 104.2 | 67.0 |
| Tablet 6 | 106.3 | 74.1 |
| Average | 105.2 | 74.0 |

Similar to the results shown in Tables 5-7 of Example 5, minocycline released more quickly than rifampin. However, rifampin released more slowly from the composition shown Example 7 than in the composition shown in Example 5.

### Example 8

Tablets with a composition as listed in Table 2 were compressed using a rotary tablet press, as described with respect to Example 3. Table 16 shows content uniformity based on a quantitative assay performed using HPLC and other standard methods. Assay results for minocycline resulted in recovery above the theoretical maximum of 100%.

TABLE 16

| | Minocycline | Rifampin |
|---|---|---|
| | assay | assay |
| | (%) | (%) |
| Tablet 1 | 111.1 | 85.9 |
| Tablet 2 | 106.4 | 87.2 |
| Tablet 3 | 111.7 | 87.2 |
| Average | 109.7 | 85.5 |

Tables 17-19 illustrate results of dissolution tests of tablets that included the composition shown in Table 2 in a 10 mM sodium phosphate buffer solution with a pH of about 6.0. Table 17 shows results after about 15 minutes, Table 18 shows results after about 30 minutes, and Table 19 shows results after about 35 minutes. As Tables 17-19 show, rifampin release was generally slower than minocycline release.

TABLE 17

| | Minocycline | Rifampin |
|---|---|---|
| | release | release |
| | (%) | (%) |
| Tablet 1 | 110.8 | 60.0 |
| Tablet 2 | 100.5 | 43.2 |
| Tablet 3 | 101.4 | 54.9 |
| Average | 104.3 | 52.7 |

TABLE 18

| | Minocycline | Rifampin |
|---|---|---|
| | release | release |
| | (%) | (%) |
| Tablet 1 | 109.6 | 70.7 |
| Tablet 2 | 104.5 | 72.2 |
| Tablet 3 | 105.5 | 65.2 |
| Average | 106.5 | 69.4 |

TABLE 19

| | Minocycline | Rifampin |
|---|---|---|
| | release | release |
| | (%) | (%) |
| Tablet 1 | 111.0 | 74.1 |
| Tablet 2 | 106.4 | 76.5 |
| Tablet 3 | 107.5 | 71.7 |
| Average | 108.3 | 74.1 |

Again, the results shown in Tables 17-19 illustrate that rifampin released at a slower rate than minocycline.

### Example 9

Tables 20-29 illustrate results of dissolution tests of tablets that included the composition shown in Table 3 in a 10 mM sodium phosphate buffer solution with a pH of about 6.0. Table 20 shows results after about one (1) hour, Table 21 shows results after about two (2) hours, Table 22 shows results after about three (3) hours, Table 23 shows results after about four (4) hours, Table 24 shows results after about six (6) hours, Table 25 shows results after about eight (8) hours, Table 26 shows results after about twelve (12) hours, Table 27 shows results after about sixteen (16) hours, Table 28 shows results after about twenty (20) hours, and Table 29 shows results after about twenty-four (24) hours. As Tables 20-29 show, rifampin release was generally between about 10% and about 20% slower than minocycline release. After twenty-four hours, neither minocycline nor rifampin was fully released.

TABLE 20

| | Minocycline | Rifampin |
|---|---|---|
| | release | release |
| | (%) | (%) |
| Tablet 1 | 5.2 | 0.0 |
| Tablet 2 | 4.9 | 0.0 |
| Tablet 3 | 5.4 | 0.0 |
| Tablet 4 | 5.1 | 0.0 |
| Tablet 5 | 5.1 | 0.0 |
| Tablet 6 | 5.3 | 0.0 |
| Average | 5.3 | 0.0 |

TABLE 21

| | Minocycline | Rifampin |
|---|---|---|
| | release | release |
| | (%) | (%) |
| Tablet 1 | 9.3 | 2.1 |
| Tablet 2 | 8.6 | 1.7 |
| Tablet 3 | 9.7 | 1.8 |
| Tablet 4 | 9.3 | 1.9 |
| Tablet 5 | 8.9 | 1.5 |
| Tablet 6 | 9.3 | 1.9 |
| Average | 9.2 | 1.8 |

TABLE 22

| | Minocycline | Rifampin |
|---|---|---|
| | release | release |
| | (%) | (%) |
| Tablet 1 | 15.3 | 4.4 |
| Tablet 2 | 14.4 | 19 |
| Tablet 3 | 15.7 | 2.8 |
| Tablet 4 | 16.1 | 2.4 |
| Tablet 5 | 14.0 | 1.4 |
| Tablet 6 | 15.1 | 2.1 |
| Average | 15.1 | 2.5 |

TABLE 23

| | Minocycline | Rifampin |
|---|---|---|
| | release | release |
| | (%) | (%) |
| Tablet 1 | 19.0 | 7.0 |
| Tablet 2 | 18.3 | 2.7 |
| Tablet 3 | 18.7 | 0.0 |
| Tablet 4 | 21.3 | 4.7 |
| Tablet 5 | 18.6 | 2.5 |
| Tablet 6 | 18.0 | 4.9 |
| Average | 19.0 | 3.6 |

TABLE 24

| | Minocycline | Rifampin |
|---|---|---|
| | release | release |
| | (%) | (%) |
| Tablet 1 | 26.6 | 13.8 |
| Tablet 2 | 24.7 | 7.3 |
| Tablet 3 | 26.3 | 8.8 |
| Tablet 4 | 27.0 | 9.2 |
| Tablet 5 | 23.1 | 6.8 |
| Tablet 6 | 25.5 | 9.6 |
| Average | 25.5 | 9.3 |

TABLE 25

| | Minocycline | Rifampin |
|---|---|---|
| | release | release |
| | (%) | (%) |
| Tablet 1 | 37.5 | 21.5 |
| Tablet 2 | 32.3 | 12.0 |
| Tablet 3 | 35.5 | 14.1 |
| Tablet 4 | 34.7 | 14.6 |
| Tablet 5 | 29.3 | 11.0 |
| Tablet 6 | 33.3 | 15.3 |
| Average | 33.8 | 14.8 |

TABLE 26

| | Minocycline | Rifampin |
|---|---|---|
| | release | release |
| | (%) | (%) |
| Tablet 1 | 52.3 | 38.2 |
| Tablet 2 | 46.2 | 21.5 |
| Tablet 3 | 49.0 | 28.7 |
| Tablet 4 | 47.6 | 26.4 |
| Tablet 5 | 41.5 | 20.2 |
| Tablet 6 | 49.8 | 29.0 |
| Average | 47.7 | 27.3 |

TABLE 27

| | Minocycline | Rifampin |
|---|---|---|
| | release | release |
| | (%) | (%) |
| Tablet 1 | 61.8 | 52.1 |
| Tablet 2 | 57.6 | 32.4 |
| Tablet 3 | 62.5 | 41.5 |
| Tablet 4 | 56.3 | 36.4 |
| Tablet 5 | 54.0 | 31.1 |
| Tablet 6 | 62.7 | 42.2 |
| Average | 59.1 | 39.3 |

TABLE 28

| | Minocycline | Rifampin |
|---|---|---|
| | release | release |
| | (%) | (%) |
| Tablet 1 | 67.0 | 62.9 |
| Tablet 2 | 64.8 | 45.4 |
| Tablet 3 | 70.6 | 55.8 |
| Tablet 4 | 65.2 | 49.6 |
| Tablet 5 | 62.4 | 44.3 |
| Tablet 6 | 65.7 | 56.3 |
| Average | 65.9 | 52.4 |

TABLE 29

| | Minocycline | Rifampin |
|---|---|---|
| | release | release |
| | (%) | (%) |
| Tablet 1 | 69.1 | 56.4 |
| Tablet 2 | 73.0 | 56.5 |
| Tablet 3 | 74.0 | 65.6 |
| Tablet 4 | 63.4 | 58.9 |
| Tablet 5 | 68.6 | 56.3 |
| Tablet 6 | 76.2 | 65.3 |
| Average | 70.7 | 61.3 |

Various examples have been described in the disclosure. These and other examples are within the scope of the following claims.

1. A system comprising:
an implantable medical device (IMD) implanted in a body of a patient; and
pharmaceutical implant implanted in the body of the patient proximate to the IMD, wherein the pharmaceutical implant comprises at least one excipient and a pharmaceutical, and wherein the pharmaceutical implant is configured to substantially fully dissolve within about 30 days after implantation of the pharmaceutical implant in the body of the patient.

2. The system of claim 1, wherein the pharmaceutical comprises an antimicrobial, and wherein the antimicrobial comprises at least one of an antibiotic, a fatty-acid antimicrobial salt, an antiseptic, an antimicrobial peptide, a quaternary ammonium, a heavy metal, or a heavy metal salt.

3. The system of claim 1, wherein the pharmaceutical comprises a first pharmaceutical, further comprising a second pharmaceutical different than the first pharmaceutical.

4. The system of claim 3, wherein the first pharmaceutical comprises rifampin, and wherein the second pharmaceutical comprises minocycline.

5. The system of claim 1, wherein the pharmaceutical comprises an analgesic, and wherein the analgesic comprises at least one of a pain reliever, an opioid, a narcotic, morphine, tramadol, acetaminophen, an anti-inflammatory agent, a COX-1-inhibitor, a COX-2-inhibitor, aspirin, ibuprofen, naproxen, a natural herbal compound, or a steroid.

6. The system of claim 1, wherein the pharmaceutical comprises a hemostatic agent, and wherein the hemostatic agent comprises at least one of a styptic, an antifibrinolytic, vitamin K, a blood coagulation factor, fibrinogen, thrombin, collagen, a polysaccharide, or chitosan.

7. The system of claim 1, wherein the at least one excipient comprises at least one of a binder, a disintegrant, a filler, a glidant, a lubricant, or a preservative.

8. The system of claim 7, wherein the at least one excipient comprises the binder, and wherein the binder comprises at least one of a starch, a sugar, cellulose, a modified cellulose, lactose, a sugar alcohol, or dibasic calcium phosphate.

9. The system of claim 7, wherein the at least one excipient comprises the disintegrant, and wherein the disintegrant comprises at least one of a starch, cellulose, cross-linked polyvinyl pyrrolidone, sodium starch glycolate, or cross-linked sodium carboxymethyl cellulose.

10. The system of claim 7, wherein the at least one excipient comprises the filler, and wherein the filler comprises at least one of a plant cellulose, dibasic calcium phosphate, lactose, sucrose, glucose, mannitol, sorbitol, calcium carbonate, or magnesium stearate.

11. The system of claim 7, wherein the at least one excipient comprises the glidant, and wherein the glidant comprises at least one of colloidal silicon dioxide or talc.

12. The system of claim 7, wherein the at least one excipient comprises the lubricant, and wherein the lubricant comprises at least one of polyethylene glycol, talc, silica, vegetable stearin, magnesium stearate, stearic acid, or sodium stearyl fumarate.

13. The system of claim 7, wherein the at least one excipient comprises the preservative, and wherein the preservative comprises at least one of vitamin A, vitamin C, vitamin E, retinyl palmitate, selenium, an amino acid, citric acid, sodium citrate, or a synthetic preservative.

14. The system of claim 1, wherein the pharmaceutical implant is configured to dissolve over a period of at least 24 hours.

15. The system of claim 1, wherein the pharmaceutical implant is configured to be attached to the implantable medical device, and wherein the implantable medical device is configured to have the pharmaceutical implant attached thereto.

16. The system of claim 1, wherein the implantable medical device comprises at least one of a drug pump, a pacemaker, an implantable cardioverter/defibrillator, an implantable neurostimulator, or an implantable monitoring device.

17. A kit comprising:
a pharmaceutical implant comprising at least one excipient and a pharmaceutical; and
an implantable medical device, wherein the pharmaceutical implant is configured to be attached to the implantable medical device, and wherein the implantable medical device is configured to have the pharmaceutical implant attached thereto.

18. The kit of claim 17, wherein the pharmaceutical comprises an antimicrobial, and wherein the antimicrobial comprises at least one of an antibiotic, a fatty-acid antimicrobial salt, an antiseptic, an antimicrobial peptide, a quaternary ammonium, a heavy metal, or a heavy metal salt.

19. The kit of claim 17, wherein the pharmaceutical comprises a first pharmaceutical, and wherein the pharmaceutical implant further comprises a second pharmaceutical different than the first pharmaceutical.

20. The kit of claim 17, wherein the first pharmaceutical comprises rifampin, and wherein the second pharmaceutical comprises minocycline.

21. The kit of claim 17, wherein the pharmaceutical comprises an analgesic, and wherein the analgesic comprises at least one of a pain reliever, an opioid, a narcotic, morphine, tramadol, acetaminophen, an anti-inflammatory agent, a COX-1-inhibitor, a COX-2-inhibitor, aspirin, ibuprofen, naproxen, a natural herbal compound, or a steroid.

22. The kit of claim 17, wherein the pharmaceutical comprises a hemostatic agent, and wherein the hemostatic agent comprises at least one of a styptic, an antifibrinolytic, vitamin K, a blood coagulation factor, fibrinogen, thrombin, collagen, a polysaccharide, or chitosan.

23. The kit of claim 17, wherein the at least one excipient comprises at least one of a binder, a disintegrant, a filler, a glidant, a lubricant, or a preservative.

24. The kit of claim 23, wherein the at least one excipient comprises the binder, and wherein the binder comprises at least one of a starch, a sugar, cellulose, a modified cellulose, lactose, a sugar alcohol, or dibasic calcium phosphate.

25. The kit of claim 23, wherein the at least one excipient comprises the disintegrant, and wherein the disintegrant comprises at least one of a starch, cellulose, cross-linked polyvinyl pyrrolidone, sodium starch glycolate, or cross-linked sodium carboxymethyl cellulose.

26. The kit of claim 23, wherein the at least one excipient comprises the filler, and wherein the filler comprises at least one of a plant cellulose, dibasic calcium phosphate, lactose, sucrose, glucose, mannitol, sorbitol, calcium carbonate, or magnesium stearate.

27. The kit of claim 23, wherein the at least one excipient comprises the glidant, and wherein the glidant comprises at least one of colloidal silicon dioxide or talc.

28. The kit of claim 23, wherein the at least one excipient comprises the lubricant, and wherein the lubricant comprises at least one of polyethylene glycol, talc, silica, vegetable stearin, magnesium stearate, stearic acid, or sodium stearyl fumarate.

29. The kit of claim 23, wherein the at least one excipient comprises the preservative, and wherein the preservative comprises at least one of vitamin A, vitamin C, vitamin E, retinyl palmitate, selenium, an amino acid, citric acid, sodium citrate, or a synthetic preservative.

30. The kit of claim 17, wherein the pharmaceutical implant is configured to substantially fully dissolve within about 30 days of implant.

31. The kit of claim 17, wherein the pharmaceutical implant is configured to dissolve over a period of at least 24 hours.

32. The kit of claim 17, wherein the implantable medical device comprises at least one of a drug pump, a pacemaker, an implantable cardioverter/defibrillator, an implantable neurostimulator, or an implantable monitoring device.

33. A pharmaceutical implant comprising:
at least one excipient;
minocycline; and
rifampin, wherein the pharmaceutical implant is configured to be implanted in a body of a patient and substantially fully dissolve within about 30 days of implantation.

34. The pharmaceutical implant of claim 33, wherein the at least one excipient comprises at least one of a binder, a disintegrant, a filler, a glidant, a lubricant, or a preservative.

35. The pharmaceutical implant of claim 34, wherein the at least one excipient comprises the binder, and wherein the binder comprises at least one of a starch, a sugar, cellulose, a modified cellulose, lactose, a sugar alcohol, or dibasic calcium phosphate.

36. The pharmaceutical implant of claim 34, wherein the at least one excipient comprises the disintegrant, and wherein the disintegrant comprises at least one of a starch, cellulose, cross-linked polyvinyl pyrrolidone, sodium starch glycolate, or cross-linked sodium carboxymethyl cellulose.

37. The pharmaceutical implant of claim 34, wherein the at least one excipient comprises the filler, and wherein the filler comprises at least one of a plant cellulose, dibasic calcium phosphate, lactose, sucrose, glucose, mannitol, sorbitol, calcium carbonate, or magnesium stearate.

38. The pharmaceutical implant of claim 34, wherein the at least one excipient comprises the glidant, and wherein the glidant comprises at least one of colloidal silicon dioxide or talc.

39. The pharmaceutical implant of claim 34, wherein the at least one excipient comprises the lubricant, and wherein the lubricant comprises at least one of polyethylene glycol, talc, silica, vegetable stearin, magnesium stearate, stearic acid, or sodium stearyl fumarate.

40. The pharmaceutical implant of claim 34, wherein the at least one excipient comprises the preservative, and wherein the preservative comprises at least one of vitamin A, vitamin C, vitamin E, retinyl palmitate, selenium, an amino acid, citric acid, sodium citrate, or a synthetic preservative.

41. The pharmaceutical implant of claim 33, wherein the pharmaceutical implant is configured to dissolve over a period of at least 24 hours.

42. A method comprising:
implanting a pharmaceutical implant in a body of a patient, wherein the pharmaceutical implant comprising an excipient and a pharmaceutical, and wherein the pharmaceutical implant is configured to substantially fully dissolve within about 30 days after implantation of the pharmaceutical implant in the body of the patient.

43. The method of claim 42, further comprising implanting an implantable medical device in the body of the patient proximate to the pharmaceutical implant.

44. The method of claim 43, further comprising attaching the pharmaceutical implant to the implantable medical device.

45. The method of claim 44, wherein attaching the pharmaceutical implant comprises adhering the pharmaceutical implant to the implantable medical device.

46. The method of claim 43, wherein implanting the pharmaceutical implant comprises implanting the pharmaceutical implant disposed in a depression in a surface of the implantable medical device.

47. The method of claim 42, wherein the pharmaceutical implant is configured to fully dissolve between 3 days and 7 days from implant.

48. The method of claim 42, wherein the pharmaceutical comprises an antimicrobial, and wherein the antimicrobial comprises at least one of an antibiotic, a fatty-acid antimicrobial salt, an antiseptic, an antimicrobial peptide, a quaternary ammonium, a heavy metal, or a heavy metal salt.

49. The method of claim 42, wherein the pharmaceutical comprises a first pharmaceutical, further comprising a second pharmaceutical different than the first pharmaceutical.

50. The method of claim 49, wherein the first pharmaceutical comprises rifampin, and wherein the second pharmaceutical comprises minocycline.

51. The method of claim 42, wherein the pharmaceutical comprises an analgesic, and wherein the analgesic comprises at least one of a pain reliever, an opioid, a narcotic, morphine, tramadol, acetaminophen, an anti-inflammatory agent, a COX-1-inhibitor, a COX-2-inhibitor, aspirin, ibuprofen, naproxen, a natural herbal compound, or a steroid.

52. The method of claim 42, wherein the pharmaceutical comprises a hemostatic agent, and wherein the hemostatic agent comprises at least one of a styptic, an antifibrinolytic, vitamin K, a blood coagulation factor, fibrinogen, thrombin, collagen, a polysaccharide, or chitosan.

53. The method of claim 42, wherein the at least one excipient comprises at least one of a binder, a disintegrant, a filler, a glidant, a lubricant, or a preservative.
